(19) United States
(12) Patent Application Publication
(10) Pub. No.: US 2013/0137859 A1
(43) Pub. Date: May 30, 2013

(54) METHODS OF CRYSTALLISING PERFORIN

(71) Applicants: James C. Whisstock, Murrumbeena (AU); Ruby H.P. Law, Caulfield (AU); Joseph Albert Trapani, Camberwell (AU); Ilia Voskoboinik, Eltham (AU)

(72) Inventors: James C. Whisstock, Murrumbeena (AU); Ruby H.P. Law, Caulfield (AU); Joseph Albert Trapani, Camberwell (AU); Ilia Voskoboinik, Eltham (AU)

(73) Assignee: MONASH UNIVERSITY, Clayton, VIC (AU)

(21) Appl. No.: 13/698,472

(22) PCT Filed: May 17, 2011
(86) PCT No.: PCT/AU2011/000578

§ 371 Date: Jan. 25, 2013

(30) Foreign Application Priority Data
May 17, 2010 (AU) 2010902124

(51) Int. Cl. C07K 14/47

(52) U.S. Cl. CPC C07K 14/4728
USPC 530/419; 530/418; 530/350

(57) ABSTRACT

A method for isolating crystals of perforin including the step of: crystallising perforin from solution at pH 6.4 to 8.0 and 20±5° C.

### FIELD OF INVENTION

The present invention relates to a method of crystallising perforin. The present invention further relates to crystals produced by the method and their use, particularly for the identification of active-sites for drug therapy.

In one embodiment the present invention relates to a method of crystallising perforin and of stabilising the crystals so that data collection is possible, for example, by x-ray crystallography.

### BACKGROUND ART

It is to be appreciated that any discussion of documents, devices, acts or knowledge in this specification is included to explain the context of the present invention. Further, the discussion throughout this specification comes about due to the realisation of the inventor and/or the identification of certain related art problems, by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

Natural killer cells and cytotoxic T-lymphocytes accomplish the critically important function of killing virus-infected and neoplastic cells by releasing the pore-forming protein perforin and granzyme proteases into the immunological synapse. Perforin, a 67 kDa multidomain protein, oligomerises to form pores that deliver pro-apoptopic granyzmes through the target cell plasma membrane into the cytosol¹⁻⁶. The importance of perforin is highlighted by the fatal consequences of congenital perforin deficiency, with over 50 different perforin mutations linked to Familial Hemophagocytic Lymphocytosis (Type 2 FHL)⁷.

Perforin is fundamental for the human immune response, where it eliminates virally infected and pre-cancerous cells. The overactivity of perforin is also central to certain diseases (e.g. cerebral malaria, inflammatory diseases, type I diabetes and transplant rejection). Hence this molecule is an important drug target. However, in the past perforin has proved difficult to isolate as good quality crystals. In particular it has proved difficult to isolate crystals of perforin that are pure enough for analytical use. In particular it has not hitherto been possible to isolate crystals of sufficient purity for x-ray crystallographic analysis.

Accordingly, there is a need for a method of isolating perforin crystals suitable for structural and other analysis. This is important for obtaining greater understanding of the mechanism of perforin pore formation. In particular this is critical to obtaining a structural model for perforin that can be functionally characterised and validated as a drug target.

### SUMMARY OF INVENTION

An object of the present invention is to provide a method of isolating perforin crystals.

A further object of the present invention is to provide a method of isolating perforin crystals and of stabilizing the crystals so that data collection is possible, for example by x-ray crystallography.

A further object of the present invention is to use the structure to elucidate the mechanism of perforin pore formation.

A further object of the present invention is to provide functional characterisation of new perforin active-site targets for therapeutics.

A further object of the present invention is to alleviate at least one of the problems associated with perforin drug development using the related art.

It is an object of the embodiments described herein to overcome or alleviate at least one of the above noted drawbacks of related art methods or to at least provide a useful alternative to related art methods.

In a first aspect of embodiments described herein there is provided a method for isolating crystals of perforin comprising the step of crystallising perforin from solution at pH 6.4 to 8.0 and 20±5° C. more preferably 20±2° C. The crystals are typically of greater purity than the starting material.

Typically the crystallisation step includes (i) solubilising the perforin, and (ii) adding crystallisation solution that induces precipitation of crystals.

Preferably the concentration of perforin in the solution of step (i) is between 2.5 and 3.5 mg/ml of solution. The perforin is dissolved in a first buffer solution, comprising for example, 50 mM TrisHCl and optionally other components such as glycerol, Na azide, NaCl and Complete Protease Inhibitor Cocktail Tablet without EDTA. The buffer capacity is between 6.4 and 8.0, preferably 7.2.

The crystallisation solution of step (ii) may comprises for example, 0.5M to 0.75M Na acetate and 1.0M imidazole pH 6.5 to 7.5. Alternatively it may for example includes a mixture of tri-sodium citrate, polyethylene glycol 4000, 13 to 17% and ammonium acetate at a pH 5 to 6.5. Typical drop ratios would be, for example 1.5 (perforin) to 1 (crystallisation solution), 1.7 to 1 or 2 to 1 etcetera. This solution may also be an important intermediate in other methods of purification or other reactions/preparative methods.

Crystallisation is preferably carried out a temperature of 20±5° C. more preferably 20±2° C. for up to 4 to 8 weeks. Streak seeding with perforin crystals may be used.

If crystals are required for data collection, such as by x-ray crystallography, the method includes the additional step of flash freezing. Flash freezing includes techniques such as freezing using liquid N₂, or putting the crystals of step (ii) in a cryostream. This technique reduces the crystals from room temperature to a very low temperature, such as −173° C. (100K) when N₂ is used for flash freezing.

Optionally the crystals will be combined with a cryoprotectant. Suitable cryoprotectants include, but are not limited to glycerol, 2-Methyl-2,4-pentanediol (MPD), polyethylene glycol 400, ethylene glycol, paratone-N, sucrose or any other suitable cryoprotectant known to the person skilled in the art. Typically the cryoprotectant is present in a concentration of about 15% to about 25%, typically 25%.

The perforin used in the method of the present invention may be expressed and obtained by any suitable method known to the person skilled in the art. In a particularly preferred embodiment the perforin is a non-oligomerising perforin mutant (such as R213E, D191 or E343), or any mutant that improves crystallisation that is designed by virtue of access to structural coordinates disclosed herein in Table A. Non-mutant perforin molecules have a tendency to aggregate—a tendency that is less apparent for mutant perforin.

In a second aspect of embodiments described herein there is provided a method for isolating perforin crystals comprising the steps of:
(i) subjecting expressed perforin to an initial purification step, and
(ii) crystallising the perforin from solution at pH 6.4 to 7.5 and 20±5° C.

The initial purification step may be carried out by any convenient method known to the person skilled in the art. In a particularly preferred embodiment the initial purification is carried out by chromatographic separation, such as metal affinity chromatography and size exclusion chromatography. The purification will typically include the use of a buffer solution for elution of the perforin from the chromatography column. When metal affinity chromatography is used, the eluent may include imidazole. When size exclusion chromatography is used, preferably the eluent has a buffering capacity between pH 6.4 and 8.0. For example, the first buffer referred to above (comprising TrisHCl) can be used as eluent.

In a third aspect of embodiments described herein there is provided a method for isolating perforin crystals comprising the steps of:
(i) subjecting expressed perforin to an initial purification step in the presence of a buffer having buffering capacity between pH 6.4 and 8.0,
(ii) crystallising the perforin from solution at pH 6.4 to 7.5 and 20±5° C. and optionally,
(iii) flash freezing the crystals, optionally in the presence of a cryoprotectant.

Many analysis methods operate on the principal of directing a beam of intense energy at a sample, then detecting the beam as it reflects, deflects or passes through the sample. One of the problems with this type of analysis is that the energy from the beam can cause disintegration of the sample, detracting from the quality and accuracy of the detection and analysis results. For example, x-ray analysis of perforin has not hitherto been possible because the perforin crystal would disintegrate in the x-ray beam before sufficient data could be collected to calculate the molecular structure. It has now been found that x-ray damage during data collection can be minimised by soaking perforin crystals in a saturated solution of heavy metal, for a period of time (from 12 hours to one week) prior to treatment with a cryo-protectant containing crystallization solution plus glycerol (15-25%) for data collection. Preferred heavy metals for inclusion in the soaking solution include the heavy metals Hg, Ir, Pt or Au as a complex. Ethylmercury phosphate is particularly suitable. The precise nature of the protection provided by back-soaking is unclear. Without wishing to be bound by theory if is postulated that the di-sulphide bonds in the perforin protein are somehow stabilised and rendered less likely to break.

Accordingly, in a fourth aspect of embodiments described herein there is provided a method for isolating perforin crystals comprising the steps of:
(i) crystallising perforin from solution at pH 6.4 to 7.5 and 20±5° C.
(ii) back-soaking the perforin crystals from step (i) in a heavy metal protectant solution, and optionally
(iii) flash freezing the crystals, optionally in the presence of a cryoprotectant.

Perforin prepared according to the present invention may be derivatised. In particular, perforin may be derivatised with Hg, Ir and Iodine, using, for example, ethylmercury phosphate, ammonium hexachloroiridate(III) and elemental iodine. The use of derivatives, particularly heavy metal derivatives is particularly useful in x-ray crystallographic analysis of proteins because it facilitates phasing of a crystal structure. Typically at least three derivatives are required to obtain enough information for phasing and to properly fix structural coordinates.

### Use of the Purified Perforin Crystals

In a fifth aspect of the embodiments described herein there is provided a method of facilitating crystallisation using a non-oligomerising perforin mutant or any mutant designed with reference to the coordinates of Table A.

A sixth aspect of the embodiments described herein relates to perforin crystals prepared according to the method of the present invention. In a preferred embodiment the perforin crystals or derivatives thereof consist of a primitive orthorhombic P2₁2₁2₁ space group and having unit cell dimensions chosen from the group comprising:
a=78.77±3.0 Å, b=110.22±3.0 Å and c=140.88±3.0 Å,
a=79.02±3.0 Å, b=112.31±3.0 Å and c=139.76±3.0 Å,
a=78.05±3.0 Å, b=108.13±3.0 Å and c=140.99±3.0 Å,
a=77.35±3.0 Å, b=104.58±3.0 Å and c=141.66±3.0 Å, or
a=78.60±3.0 Å, b=109.91±3.0 Å and c=140.84±3.0 Å.

A seventh aspect of the embodiments described herein relates to the use of a perforin purified according to the method of the present invention. In particular it is intended to include the use of said perforin for soaking in potential inhibitors, activators, or drug fragments that could be used to build drugs.

Perforin is a thin “key-shaped” molecule, comprising an N-terminal Membrane Attack Complex Perforin-like (MACPF)/Cholesterol Dependent Cytolysin (CDC) domain^{8,9} followed, by an EGF domain that, together with the extreme C-terminal sequence, forms a central shelf-like structure. A C-terminal C2 domain mediates Ca²⁺ dependent membrane binding, and structural comparisons suggest a novel conformational rearrangement takes place within this domain in response to metal ion binding.

Crystals prepared according to the method of the present invention have proved suitable for use in x-ray crystallography. In particular, crystals prepared according to the method of the present invention have been used to provide a structural model for native versions of the perforin monomer solus, and Hg, Ir and Iodine derivatives of perforin. The use of these structural models has been validated. The structural model, having been validated, can be used for the identification of novel classes of therapeutics using high-throughput chemical screening and medicinal chemistry methods.

In particular the structural model provides insight into how perforin initially interacts with membranes via the C2 domain and how interaction with lipids or membranes may triggering of conformational change in the membrane binding region. The similarilty with CDCs also suggests that perforin oligomerises via the flat faces—accordingly, in developing perforin inhibitors both flat faces of perforin would be attractive regions to target to prevent oligomerisation. Together the structural model also provides a means of identifying drug target sites such as cavities or pores. Optimally this will lead to identification of binding sites that can be used to interfere with lipid binding or oligomerisation.

In an eighth aspect of embodiments described herein there is provided a method for screening molecules or molecular complexes for anti-perforin activity comprising the steps of:
(a) purifying perforin according to the method of the present invention,
(b) characterising an active site cavity;
(c) identifying candidate molecules or molecular complexes that interact with at least part of the active site cavity; and
(d) obtaining or synthesising said candidate molecule or molecular complex.

One of the advantages of using a structure based model as a drug target is that it has a high degree of specificity, that is, the model makes it possible to choose or design a molecule of molecular complex that coordinates to perforin or its derivatives, but does not adversely affect other molecules that may be beneficial, or essential to a host.

In a ninth aspect of embodiments described herein there is provided a method for screening molecules or molecular complexes for anti-perforin activity comprising the steps of:
(i) purifying perforin according to the method of the present invention;
(ii) characterising the pore of the perforin;
(iii) identifying candidate molecules or molecular complexes that interact with at least part of the pore; and
(iv) obtaining or synthesising said candidate molecule or molecular complex.

In particular, step (ii) may include identifying molecules or molecular complexes that interact with one or more of the residues involved at the pore.

The present invention further provides an active binding site or active binding site cavity in the perforin structure or its derivatives as well as methods for designing or selecting molecules or molecular complexes for use as anti-perforin drugs using information about the crystal structures disclosed herein. The present invention further provides anti-perforin drugs or drug candidates designed or selected according to said method.

In a preferred embodiment, the methods, drugs or drug candidates of the present invention are suitable for modulating perforin, to inhibit at least part of its activity, more preferably all of its activity. In a particularly preferred embodiment, the methods, drugs or drug candidates of the present invention are suitable for modulating native forms of perforin and their Hg, Ir, I derivatives to inhibit at least part of their activity, more preferably all of their activity. In a particularly preferred embodiment, the inhibition will stop perforin membrane binding, perforin oligomerisation, perforin conformational change, cell degradation (for example through lysis), cell destruction or perforin mediated delivery of a cytotoxic protein such as a granyzme or any other toxic molecule (proteinaceous or otherwise) to a target cell. In a further preferred embodiment the drug template of the present invention includes use of the C-terminus of perforin, which is anticipated to line the pore lumen, could be changed or utilised to preferentially deliver toxic species (protein or otherwise) to the cell cytoplasm.

Other aspects and preferred forms are disclosed in the specification and/or defined in the appended claims, forming a part of the description of the invention.

In essence, embodiments of the present invention stem from the realization that a new method can be used to obtain pure perforin. Furthermore, it has been realised that perforin can be stabilised for use in analysis techniques hitherto unsuited for perforin. Structural information regarding the active site of perforin can be used to identify and guide development of inhibitors that have activity.

Advantages provided by the present invention comprise the following:
provision of a method that can isolate perforin that is suitable for structural analysis,
provision of perforin that is suitably pure for creation of a drug target model,
a new method for providing perforin for use in anti-perforin drug screening, design and development,
the ability to use perforin (or any mutant thereof) crystals for preparing or identifying potential inhibitors, activators, or drug fragments and the building of new drugs,
the ability, to use perforin for creation of structures useful in (i) the development of inhibitors that block the likely mechanism of conformational changes in CH1, CH2 and the C2 domain of perforin, and (ii) the development of compounds that interfere with perforin function by binding or interfering with the glycosylation present on the human molecule, or by interacting with the C-terminus (for example, therapeutic monoclonals that target the C-terminal peptide, or any region of the molecule that would be anticipated to line the pore) and (iii) the identification of molecules that may stabilise the fold of perforin polymorphisms that result in instabity (e.g. A91V) and that take advantage of the structural data.
new applications of perforin as a nanotechnological reagent, particular with regards to including C-terminally linked moieties (whether these be enyzmes, nanoparticles or other medically or commercially useful reagents), or molecules that clearly take advantage of the perforin mechanism of pore formation as revealed by the structural data.

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other embodiments of the present application may be better understood by those skilled in the relevant art by reference to the following description of embodiments taken in conjunction with the accompanying drawings, which are given by way of illustration only, and thus are not limitative of the disclosure herein, and in which:

FIG. 1 illustrates the structure of perforin monomers.

FIGS. 1(a) and 1(b)—The MACPF domain is in dark grey; CH1 and CH2 (labelled) are lighter grey. The EGF, C2 domain and the C-terminal region are labelled. The region outlined by the dashed box is the shelf region. Two Ca²⁺ atoms are shown in grey spheres. The MACPF domain contains three N-linked oligosaccharides, one of which (attached to N204) is visible in electron density and is labelled (NAG). The positions of the two other oligosaccharides (attached to N375 and N548) are indicated by spheres and are labelled In b) the position of the R213E mutation is shown in stick.

FIG. 2 illustrates the crystal structure of a perforin and the representative CDC PFO.

FIG. 2(a)—Crystal structure of perforin

FIG. 2(b)—the representative CDC PFO⁵³. In 2a) and b) the homologous C2 and Ig domains labelled—note the MACPF domain faces the opposite direction.

FIGS. 2(c) and 2(d)—A schematic illustrating CDC membrane insertion mechanism. The two clusters of helices CH1 and CH2 (also known as Transmembrane Helices-1 [TMH] and -2), unwind to insert into membranes as amphipathic β-strands (d), with the hydrophobic side facing the membrane, and the hydrophilic face lining the interior of the pore.

FIG. 3—illustrates binding sites in the C2 domain.

FIG. 3(a)—Cartoon showing the position of the three Calcium binding regions (CBR1-3). The first strand of the C2 domain (dark grey) finishes in CBR1 and is linked to CH2 at its N-terminus via the C241/C407 disulphide bond. Ca²⁺ atoms are in grey spheres.

FIG. 3(b)—The base of the perforin C2 domain. The Ca²⁺ binding site with the functionally important (as determined by mutagenesis studies¹¹; metal ion coordinating residues are in stick. The site I calcium atom is coordinated by residues D435 and D483 as well as the carbonyl oxygen of A484. A second Ca²⁺ atom is located on the other side of CBR3. D429 is located ˜8 Å away from the Ca²⁺ binding site. Aromatic residues (W453, W488, Y430 and Y486 [the sidechain of which is partly disordered]) are seen at the base.

FIG. 3(c)—Superposition of the perforin C2 domain with MUNC-13, its closest structurally characterised homologue (pdb identifier 3KWU; 26% identical)¹⁴. The position of CBR1 and the site II Ca²⁺ in MUNC-13 is labelled. Residue D705, which is equivalent to D429 in perforin coordinates both site I and site II Ca²⁺ atoms in the MUNC-13 structure. In perforin, D429 would have to undergo a ˜8 Å repositioning (arrowed) in order to coordinate Ca²⁺ in a canonical fashion.

FIG. 3(d)—a schematic illustrating the Asp/Ca²⁺ interactions, CBR1-3 are labelled. Residues in black interact with Ca²⁺ (dashed black lines). Light grey residues are conserved Ca²⁺ binding residues that coordinate metal ions in other C2 domain structures^{14,15}, in particular, the site II Ca²⁺ (dashed cyan sphere) is located centrally between CBR1 and CBR3.

FIG. 4 illustrates sequence alignment of MUNC-13 and perforin C2. PSI-BLAST¹⁶ and DALI¹⁷ searches reveal that the MUNC-13 C2 domain is the closest structurally characterised homologue. An arrow indicates a single amino acid deletion in perforin in the loop preceding D429. Canonical Ca²⁺ binding residues are shaded.

### DETAILED DESCRIPTION

The sequence similarity between perforin and complement components C6-C9 of the Membrane Attack Complex strongly suggests that two major branches of mammalian immunity utilize a pore forming “MACPF” fold as the final weapon that mediates target cell death^{10,16-20}. Recent structural studies on Plu-MACPF⁸ and human complement C8α^{9,21} surprisingly revealed that vertebrate MACPF proteins are homologous to bacterial CDCs like Perfringolysin O (PFO)^{8,9,11,21-23}. In addition to the MACPF domain, perforin contains a Ca²⁺-dependent, membrane binding C2 domain homologous to the membrane binding immunoglobulin domain of CDCs²⁴. However, without the structures of a complete lytic MACPF protein and a MACPF pore, the mechanisms of perforin function and dysfunction remain unclear. To address these issues, the 2.75 Å resolution structure of mouse perforin (an oligomerisation impaired variant, R213E)¹¹ was determined.

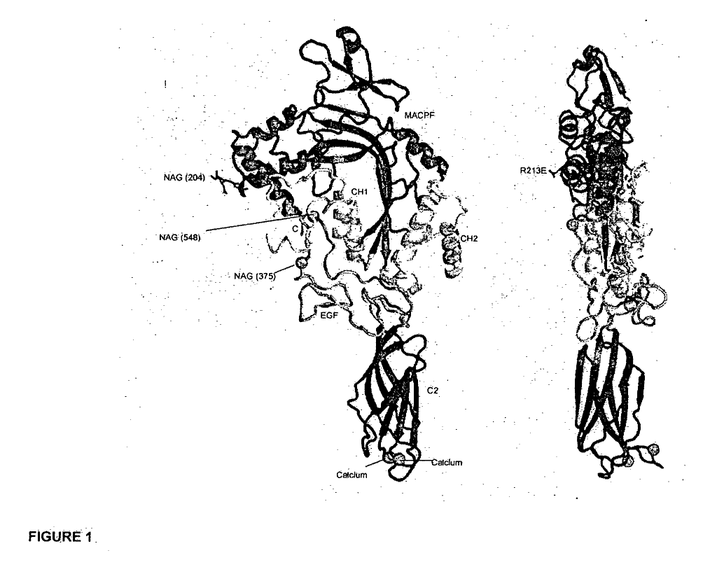

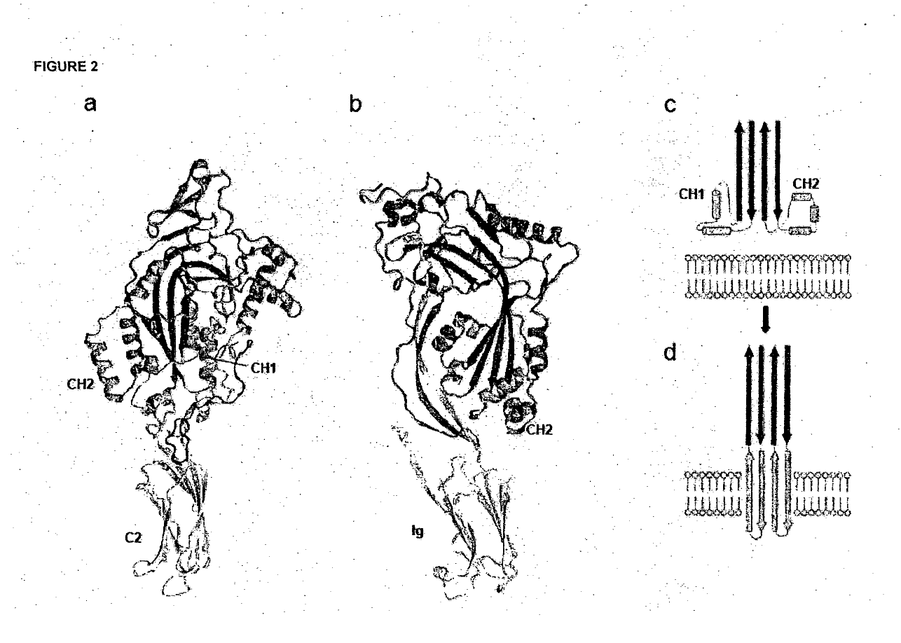

The perforin monomer structure (FIG. 1a, FIG. 2 and Table 1) roughly resembles the shape of bacterial CDCs, with a long dimension of 125 Å.

TABLE 1
Data collection, phasing and refinement statistics (MIR)

| | Derivatives | | | | |
|---|---|---|---|---|---|
| | Native 1 | Hg | Ir | I | Native 2 |
| Data collection | | | | | |
| Space group | P2₁2₁2₁ | P2₁2₁2₁ | P2₁2₁2₁ | P2₁2₁2₁ | P2₁2₁2₁ |
| Cell dimensions | | | | | |
| a, b, c (Å) | 78.77, 110.22, | 79.02, 112.31, | 78.05, 108.13, | 77.35, 104.58, | 78.60, 109.91, |
| | 140.88 | 139.76 | 140.99 | 141.66 | 140.84 |
| αβγ (°) | 90, 90, 90 | 90, 90, 90 | 90, 90, 90 | 90, 90, 90 | 90, 90, 90 |
| Resolution (Å) | 19.95 (3.00) | 19.80 (3.35) | 19.80 (3.5) | 20.07 (3.5) | 19.93 (2.75) |
| R_{merge}/Rₚᵢₘ | 11.1 (70.3)/ | 20.9 (69.4)/ | 16.2 (57.9)/ | 23.8 (111.9)/ | 11.5 (110.4)/ |
| | 5.7 (36.7) | 8.5 (28.1) | 5.4 (19.5) | 7.2 (34.1) | 4.0 (40.7) |
| I/σI | 13.4/3.1 | 11.5/4.1 | 18.8/7.4 | 13.3/3.9 | 17.5/2.7 |
| Completeness (%) | 99.7 (100) | 99.5 (100) | 99.5 (100) | 99.5 (100) | 99.7 (100) |
| Redundancy | 8.6 (8.7) | 12.7 (13.0) | 18.5 (18.9) | 22.5 (22.7) | 16.5 (16.9) |
| Anomalous | 99.8 (100) | 100 (99.9) | 99.6 (100) | 99.7 (100) | 99.8 (100) |
| Redundancy | | | | | |

| | |
|---|---|
| Refinement | |
| Resolution (Å) | 19.91 (2.75) |
| No. reflections | 30629/1676 |
| (work/free) | |
| R_{work/}R_{free} | 19.61/21.68 |
| No. atoms | |
| Protein | 4063 |
| Ligand/ion | 40 |
| Water | 279 |
| B-factors | |
| Protein | 80.724 |
| Ligand/ion | 119.924 |
| Water | 72.2 |
| R.m.s deviations | |
| Bond lengths (Å) | 0.007 |
| Bond angles (°) | 0.98 |

*Highest resolution shell is shown in parenthesis.

A central feature of the perforin MACPF domain is a bent and twisted four-stranded β-sheet flanked by two clusters of α-helices, termed CH1 and CH2 (FIG. 1a). In CDCs, the regions equivalent to CH1 and CH2 unwind to insert into membranes as amphipathic β-strands^{25,26} (FIG. 2). In perforin, CH1 is loosely held between the central sheet, the C-terminal α-helix and the disulphide constrained EGF-like fold that follows the MACPF domain (FIG. 1a-c). At the end of the EGF domain a disulphide bond (C407/C241) is formed with the first helix of CH2 (FIG. 1c). Interestingly, the EGF domain is intimately associated with the extreme C-terminal sequence (residues 524-551). Together these form a continuous shelf on which the MACPF sits (FIG. 1a-c) and beneath which hangs a type II (rather than the predicted type I)²⁷ C2 domain (FIGS. 1a, b). The close proximity of the N- and C-termini of the C2 domain and structural continuity of the shelf region suggest that the C2 domain may have been inserted into an ancestral MACPF protein that contained a C-terminal array of small disulphide constrained structures (FIG. 1c). Single particle EM maps of wild type mouse perforin monomers are in good agreement with the perforin crystal structure (FIGS. 1d, e). In addition, the EM maps reveal variable angles between the C2 and MACPF domains, suggesting the shelf region contains a hinge point. In support of this, B-factor analysis suggests the EGF domain (as well as parts of CH1 and CH2) is extremely flexible.

Many C2 domain proteins bind membranes in a Ca²⁺ dependent fashion. The C2 fold can coordinate up to four Ca²⁺ atoms (at sites I-IV); these can promote conformational change within the Ca²⁺ binding loops^{14,24}. Furthermore, the metal ions themselves may interact with lipid head groups^{14,24}. The C2 domain of perforin is central to regulation of its activity; low concentrations of Ca²⁺ in the granule prevent premature triggering of perforin activity, whereas upon granule exocytosis higher extracellular Ca²⁺ promotes membrane binding^{1-8,25-27} as well as conformational change and self-association^{25,26} (hence crystallisation experiments were performed without added Ca²⁺).

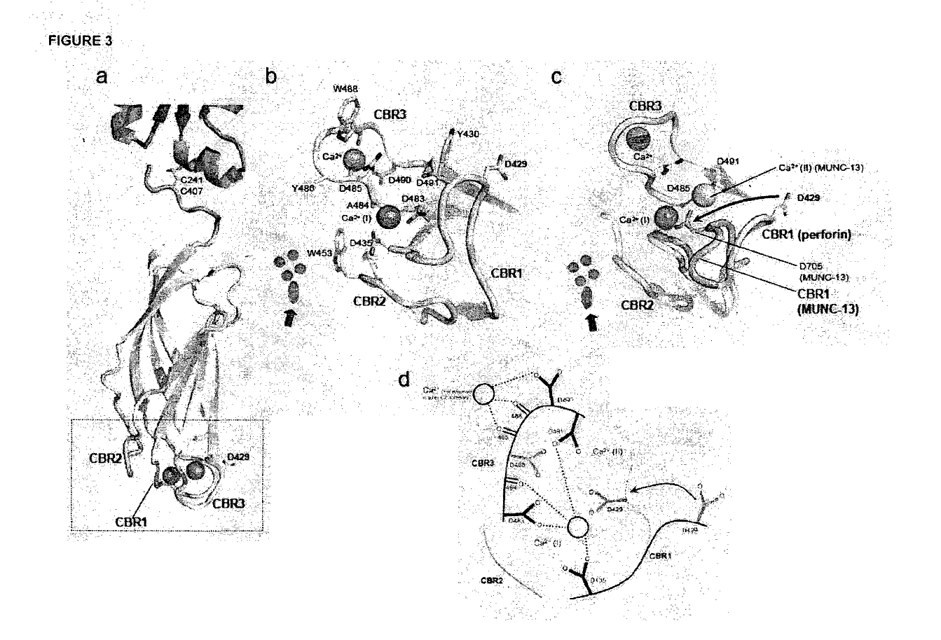

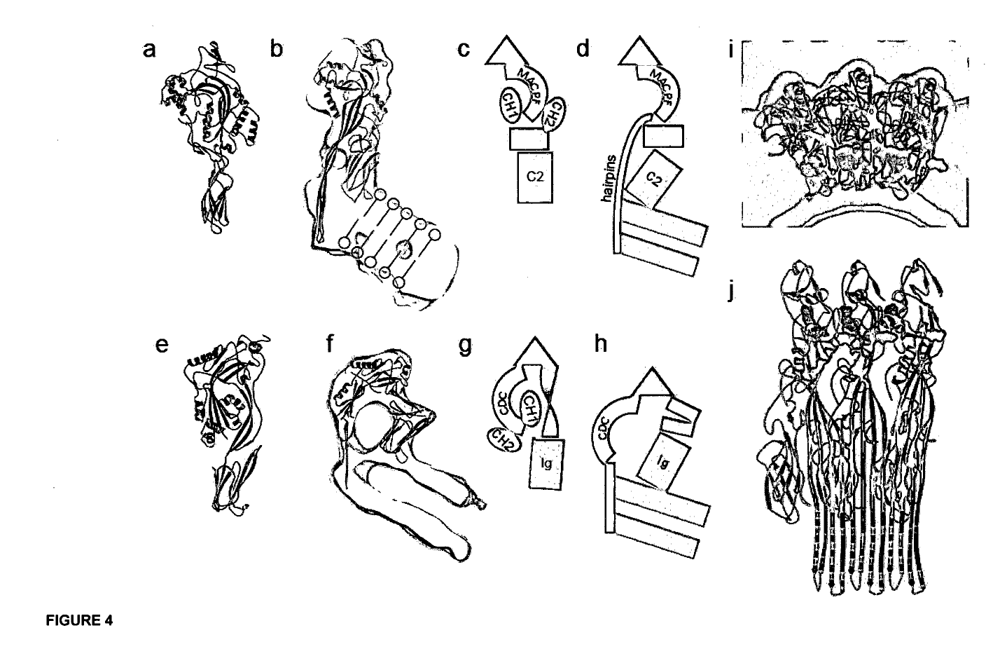

Inspection of the perforin structure suggests how Ca²⁺ binding by its C2 domain effects membrane attachment. We observed one Ca²⁺ atom canonically coordinated in the site I position between the 3 Calcium Binding Regions (CBR1-3) of the C2 domain (FIGS. 3a, b). A second Ca²⁺ atom is coordinated outside of CBR3 by D490. This site is not conserved in other C2 domains, and D490 is not essential for perforin function²⁷ (FIG. 3b). A structural comparison between the perforin C2 domain with the MUNC-13 C2 domain (which shares 26% sequence identity)¹⁴ reveals that the site II Ca²⁺ binding site¹⁴ is unoccupied (FIG. 3b-d). Crucially, D429, a functionally essential residue²⁶ that in MUNC-13 coordinates both site I and site II Ca²⁺ ions, is located in perforin ˜8 Å away where it faces into solvent (FIG. 3b-d). The repositioning of D429 in perforin is partly due to a single amino acid deletion in the preceding loop (residues 426-428; FIG. 4). Thus, in order for D429 to play a functional role in Ca²⁺ binding, a major conformational change would be required to swing D429 round to coordinate Ca²⁺ atoms (FIG. 3b-d). In comparison to other structurally characterised C2 domains, the requirement for such a major shift in one of the canonical Ca²⁺ coordinating residues is unprecedented. However, once both site I and II Ca²⁺ atoms are bound, the perforin C2 domain will presumably be capable of interacting strongly with membranes as observed for other C2 family members¹⁴; indeed several aromatic residues at the C2 base could interact with lipid acyl groups (FIG. 3b). The requirement for a substantial conformational rearrangement in the C2 domain to coordinate the final metal ion may also underlie the unusually low affinity that perforin possesses for Ca²⁺ (200-300 μM in contrast to <5 μM for a typical intracellular C2 domain). Finally, it is interesting to note that a disulfide bridge between CH2 (C241) and a site (C407) immediately N-terminal to the C2 domain, is directly connected to the D429 loop (CBR1; FIGS. 1c and 3a). This may be important in coupling membrane binding to conformational change in pore formation.

Following Ca²⁺ mediated interactions with membranes, perforin monomers assemble into a pore with ≧100 Å diameter¹⁻⁷. Structural comparisons with CDCs suggest that perforin oligomerises via an “inside out” mechanism with the monomer orientated in the opposite direction. The implications for these data are that perforin residues that are glycosylated, as well as the perforin C-terminus, would line the pore. Accordingly, it is suggested that the perforin delivers granyzmes, which bind glycosaminoglycans³⁰, into cells through an oligosaccharide lined pore.

### Methods Summary

The methods used to obtain the results disclosed are described in the following paragraphs:

### EXAMPLE 1

Protein production and crystallography: Expression and initial purification of recombinant mouse perforin R213E, was as described¹¹, followed by size exclusion chromatography using a HiLoad 16/60 Superdex 200 pg column (GE Healthcare) in a buffer containing 50 mM Tris, 300 mM NaCl, 10% glycerol, 0.05% Na azide, pH 7.2 plus Complete Protease Inhibitor Cocktail Tablet without EDTA (Roche Applied Science). Purified perforin (3 mg/ml) was crystallised in 0.5 M Na acetate, 0.1 M imidazole, pH 6.5 at 22° C. The crystals were flash-frozen in liquid nitrogen using 25% glycerol as the cryoprotectant. All the datasets were collected at the Australian Synchrotron MX2 beamline and were highly anisotropic (as measured by the Diffraction Anisotropy Server http://www.doe-mbi.ucla.edu/˜sawaya/anisoscale/)²⁸. These data were merged and processed using XDS²⁹, PINTLESS and SCALAR³⁰. Five percent of the datasets were flagged as a validation set for calculation of the R_{free} with neither a sigma, nor a low-resolution cut-off applied to the data. Experimental phases (See Table 1) were obtained by the MIRAS method; a native (Native1) dataset and three heavy atom derivatives (ethylmercury phosphate, ammonium hexachloroiridate(III) and iodine) were used for phasing. Experimental phasing was carried out using autoSHARP³¹; heavy atom positions were located using SHELXC/SHELXD³² and refined using SHARP³³ with resulting isomorphous (acentric) and anomalous phasing powers of 0.982 and 0.950, respectively at the resolution range 19.8-3.33 Å. The initial phases were improved by solvent flipping using SOLOMON³⁴ and density modification using DM³⁵, which dramatically increased the figure of merit (FOM) from 0.34 to 0.86,

such a large increase in FOM is probably due to the very high solvent content of the crystal (70.2%). One molecule was found per asymmetric unit and an initial model was generated using BUCCANEER³⁶. Model building was performed using COOT³⁷ while refinement was performed using PHENIX³8, REFMAC³⁹ and autoBUSTER⁴⁰. A higher resolution native dataset (Native2, 2.75 Å) was subsequently collected on a crystal soaked in 0.6M Kl and phase extension was carried out to 2.75 A using DM³⁵. Water molecules were added to the model when the R_{free} reached 30%. The recombinant perforin comprises 560 residues where the first 20 amino acids (a signal peptide) were cleaved off during secretion. Residues 21-134 and 136-547 were modelled; Pro135 in CH1 could not be built into density. The model contains two calcium ions, Ca701 and Ca702 that are 5 and 4 coordinate, respectively. Both Ca ions are in a distorted octahedral geometry. Murine perforin contains three N-linked glycosylation sites, however, density is only observed for the first N-acetylglueosamines attached to Asn204. The NAG models were made using the PRODRG server (http://davapc1.bioch.dundee.ac.uk/prodrq/). The final model also contains three glycerols, two chloride ions and four iodide ions. Crystallographic and structural analysis was performed using CCP4 suite⁴¹, WHATIF⁴² and MUSTANG⁴³ unless otherwise specified. FIGS. 1-4 were generated in part using PYMOL⁴⁵. Structural validation was performed using MolProbity⁴5. In the final structure, 2 residues (L307 and Y486) are in disallowed regions in the Ramachandran plot. The MolProbity score is 1.56 which is in 100th percentile of structures reported at this resolution. A summary of diffraction and refinement statistics can be found in Table 1. The coordinates of perforin, together with the structure factors are deposited in the protein data bank. All diffraction images are deposited in TARDIS (http://tardis.edu.au/) and are freely available.

Crystallography: Murine perforin R213E was expressed as previously described¹¹. Recombinant material was concentrated to 3 mg/ml and crystals obtained in 0.5 M Na acetate, 0.1 M imidazole, pH 6.5. A native (Native1) and three heavy atom derivatives (ethylmercury phosphate, ammonium hexachloroiridate (III) and iodine) were collected and experimental phases (Table 1) were obtained by the multiple isomorphous replacement with anomalous scattering (MIRAS). Model building was performed using coot.

### EXAMPLE 2

Preparation of perforin: Perforin protein in insect cells supernatant was dialysed intoBuffer A containing 300 mM NaCl and 50 mM NaH₂PO₄. Imidazole was then added to a final concentration of 15 mM. Perforin in the solution was partially purified by immobilised metal affinity chromatography using Ni-NTA (Qiagen). Protein bound to the Ni-NTA was eluted with Buffer B containing 270 mM imidazole 300 mM NaCl and 20 mM Tris, pH 8.0.

The partially purified product was further purified by size exclusion chromatography using a HiLoad 16/60 Superdex 200 pg column (GE Healthcare) in Buffer C containing 50 mM Tris, 300

mM NaCl, 10% glycerol, 0.05% Na azide and Complete Protease Inhibitor Cocktail Tablet (Roche Applied Science), pH 7.2. Purified perforin was concentrated to 3 mg/ml with a centrifugal filter unit (Millipore), and used immediately for protein crystallisation.

Perforin crystals were then obtained by mixing protein solution with a crystallisation solution containing 0.5-0.75 M Na acetate, 0.1 M imidazole, pH 6.5-7.5 at a range of drop ratios (e.g. 1.5 to 1; 1.7 to 1 and 2 to 1 etc.) with or without streak seeding with perforin crystals and incubate at 22° C. for 4-8 weeks. To minimize radiation damage during data collection, these crystals were soaked in the saturated solution of ethylmercury phosphate for a period of time (from 2 hours to one week) before treatment with a cryo-protectant containing crystallization solution plus glycerol (15-25%) for data collection.

### EXAMPLE 3

Alternative crystallisation solution: Perforin crystals obtained by mixing protein solution with a crystallisation solution containing tri-sodium citrate (100 mM), polyethylene glycol 4000 13 to 17% and ammonium acetate (200 mM) pH 6.5-7.5 at a range of drop ratios (e.g. 1.5 to 1; 1.7 to 1 and 2 to 1 etc.) with streak seeding with perforin crystals and incubate at 22° C. for 4-8 weeks. To minimize radiation damage during data collection, these crystals were soaked in the saturated solution of ethylmercury phosphate for a period of time (from 2 hours to one week) before treatment with a cryo-protectant containing crystallization solution plus glycerol (15-25%) for data collection.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures.

It should also be noted that where a flowchart is used herein to demonstrate various aspects of the invention, it should not be construed to limit the present invention to any particular logic flow or logic implementation.

Various embodiments of the invention may be embodied in many forms, including computer program logic for use with a processor (e.g., a microprocessor, microcontroller, digital signal processor, or general purpose computer), programmable logic for use with a programmable logic device (e.g., a Field Programmable Gate Array (FPGA) or other PLD), or any other means including any combination thereof. Computer program logic implementing all or part of the functionality where described herein may be embodied in various forms, including a source code form, a computer executable form, and various intermediate forms (e.g., forms generated by an assembler, compiler, linker, or locator).

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

### REFERENCES

1 Tschopp, J., Masson, D., and Stanley, K. K., Structural/functional similarity between proteins involved in complement- and cytotoxic T-lymphocyte-mediated cytolysis. Nature 322 (6082), 831 (1986).
2 Shinkai, Y., Takjo, K., and Okumura, K., Homology of perforin to the ninth component of complement (C9). Nature 334 (6182), 525 (1988).
3 Lichtenheld, M. G. et al., Structure and function of human perforin. Nature 335 (6189), 448 (1988).
4 Lowin, B., Hahrie, M., Mattmann, C., and Tschopp, J., Cytolytic T-cell cytotoxicity is mediated through perforin and Fas lytic pathways. Nature 370 (6491), 650 (1994).
5 Kagi, D. et al., Cytotoxicity mediated by T cells and natural killer cells is greatly impaired in perforin-deficient mice. Nature 369 (6475), 31 (1994).
6 Young, J. D., Conn, Z. A., and Podack, E. R., The ninth component of complement and the pore-forming protein (perforin 1) from cytotoxic T cells: structural, immunological, and functional similarities. Science 233 (4760), 184 (1986).
7 Voskoboinik, I., Smyth, M. J., and Trapani, J. A., Perforin-mediated target-cell death and immune homeostasis. Nat Rev Immunol 6 (12), 940 (2006).
8 Rosado, C. J. et al., A common fold mediates vertebrate defense and bacterial attack. Science 317 (5844), 1548 (2007).
9 Hadders, M. A., Beringer, D. X., and Gros, P., Structure of C8alpha-MACPF reveals mechanism of membrane attack in complement immune defense. Science 317 (5844), 1552 (2007).
10 Rossjohn, J. et al., Structure of a cholesterol-binding, thiol-activated cytolysin and a model of its membrane form. Cell 89 (5), 685 (1997).
11 Baran, K. et al., The molecular basis for perforin oligomerization and transmembrane pore assembly. Immunity 30 (5), 684 (2009).
12 Kubalek, E. W., Le Grice, S. F., and Brown, P. O., Two-dimensional crystallization of histidine-tagged, HIV-1 reverse transcriptase promoted by a novel nickel-chelating lipid. J Struct Biol 113 (2), 117 (1994).
13 Ramachandran, R., Tweten, R. K., and Johnson, A. E., The domains of a cholesterol-dependent cytolysin undergo a major FRET-detected rearrangement during pore formation. Proc Natl Acad Sci U S A 102 (20), 7139 (2005).
14 Shin, O. H. et al., Munc13 C2B domain is an activity-dependent Ca2+ regulator of synaptic exocytosis. Nat Struct Mol Biol 17 (3), 280 (2010).
15 Rizo, J. and Sudhof, T. C., C2-domains, structure and function of a universal Ca2+-binding domain. J Biol Chem 273 (26), 15879 (1998).
16 Altschul, S. F. and Koonin, E. V., Iterated profile searches with PSI-BLAST—a tool for discovery in protein databases. Trends Biochem Sci 23 (11), 444 (1998).
17 Holm, L., Kaariainen, S., Rosenstfom, P., and Schenkel, A., Searching protein structure databases with DaliLite v.3. Bioinformatics 24 (23), 2780 (2008).
18 Baran, K. et al., The molecular basis for perforin oligomerization and transmembrane pore assembly. Immunity 30 (5), 684 (2009).
19 Rosado, C. J. et al., The MACPF/CDC family of pore-forming toxins. Cell Microbiol 10 (9), 1765 (2008).
20 Slade, D. J. et al., Crystal structure of the MACPF domain of human complement protein C8 alpha in complex with the C8 gamma subunit. J Mol Biol 379 (2), 331 (2008).
21 Slade, D. J. et al., Crystal structure of the MACPF domain of human complement protein C8 alpha in complex with the C8 gamma subunit. J Mol Biol 379 (2), 331 (2008).
22 Tilley, S. J. et al., Structural basis of pore formation by the bacterial toxin pneumolysis Cell 121 (2), 247 (2005).
23 Rossjohn, J. et al., Structure of a cholesterol-binding, thiol-activated cytolysin and a model of its membrane form. Cell 89 (5), 685 (1997).
24 Hurley, J. H. and Misra, S., Signaling and subcellular targeting by membrane-binding domains. Anna Rev Biophys Biomol Struct 29, 49 (2000).
25 Shepard, L. A. et al., Identification of a membrane-spanning domain of the thiol-activated pore-forming toxin Clostridium perfringens perfringolysin O: an alpha-helical to beta-sheet transition identified by fluorescence spectroscopy. Biochemistry 37 (41), 14563 (1998).
26 Shatursky, O. et al., The mechanism of membrane insertion for a cholesterol-dependent cytolysin: a novel paradigm for pore-forming toxins. Cell 99 (3), 293 (1999).
27 Urrea Moreno, R. et al., Functional assessment of perforin C2 domain mutations illustrates the critical role for calcium-dependent lipid binding in perforin cytotoxic function. Blood 113 (2), 338 (2009).
25 Podack, E. R., Young, J. D., and Cohn, Z. A., Isolation and biochemical and functional characterization of perforin 1 from cytolytic T-cell granules. Proc Natl Acad Sci U S A 82 (24), 8629 (1985).
26 Young, J. D. et al., Functional channel formation associated with cytotoxic T-cell granules. Proc Natl Acad Sci U S A 83 (1), 150 (1986).
27 Voskoboinik, I. et al., Calcium-dependent plasma membrane binding and cell lysis by perforin are mediated through its C2 domain: A critical role for aspartate residues 429, 435, 483, and 485 but not 491. J Biol Chem 280 (9), 8426 (2005).
28 Strong, M. et al., Toward the structural genomics of complexes: crystal structure of a PE/PPE protein complex from Mycobacterium tuberculosis. Proc Natl Acad Sci U S A 103 (21), 8060 (2006).
29 Kabsch, W., Xds. Acta Crystallogr D Biol Crystallogr 66 (Pt 2), 125 (2010).
30 Evans, P., Scaling and assessment of data quality. Acta Crystallogr D Biol Crystallogr 62 (Pt 1), 72 (2006).
31 Vonrhein, C., Blanc, E., Roversi, P., and Bricogne, G., Automated structure solution with autoSHARP. Methods Mol Biol 364, 215 (2007).
32 Sheldrick, G. M., Experimental phasing with SHELXC/D/E: combining chain tracing with density modification. Acta Crystallogr D Biol Crystallogr 66 (Pt 4), 479 (2010).
33 de la Fortelle, E. and Bricogne, G., Maximum-Likelihood Heavy-Atom Parameter Refinement for Multiple Isomorphous Replacement and Multiwavelength Anomalous Diffraction Methods. Methods in Enzymology 276, 472 (1997).
34 Abrahams, J. P. and Leslie, A. G., Methods used in the structure determination of bovine mitochondrial F1 ATPase. Acta Crystallogr D Biol Crystatlogr 52 (Pt 1), 30 (1996).
35 Cowtah, K. D. and Zhang, K. Y., Density modification for macromolecular phase improvement. Prog Biophys Mol Biol 72 (3), 245 (1999).
36 Cowtan, K., The Buccaneer software for automated model building. 1. Tracing protein chains. Acta Crystallogr D Biol Crystallogr 62 (Pt 9), 1002 (2006).
37 Emsley, P., Lohkamp, B., Scott, W. G., and Cowtan, K., Features and development of Coot. Acta Crystallogr D Biol Crystallogr 66 (Pt 4), 486 (2010).
38 Adams, P. D. et al., PHENIX: a comprehensive Python-based system for macromolecular structure solution. Acta Crystallogr D Biol Crystallogr 66 (Pt 2), 213 (2010).
39 Murshudov, G. N., Vagin, A. A., and Dodson, E. J., Refinement of macromolecular structures by the maximum-likelihood method. Acta Crystallogr D Biol Crystallogr 53 (Pt 3), 240 (1997).
40 Bricogne, G. et al., BUSTER, version 2.8.0. Cambridge, United Kingdom: Global Phasing Ltd. (2009).
41 CCP4, The CGP4 suite: programs for protein crystallography. Acta Crystallogr D50, 760 (1994).
42 Vriend, G., WHAT IF: a molecular modeling and drug design program. J Mot Graph 8 (1), 52 (1990).
43 Konagurthu, A. S., Whisstock, J. C., Stuckey, P. J., and Lesk, A. M., MUSTANG: a multiple structural alignment algorithm. Proteins 64 (3), 559 (2006).
44 DeLano, W. L., The PyMOL Molecular Graphics System. DeLano Scientific LLC, Palo Alto, Calif., USA. http://www.pymol.org. (2008).
45 Davis, I. W. et al., MolProbity: all-atom contacts and structure validation for proteins and nucleic acids. Nucleic Acids Res 35 (Web Server issue), W375 (2007).

1. A method for isolating crystals of perforin comprising the step of: crystallising perforin from solution at pH 6.4 to 8.0 and 20±5° C.

2. The method for isolating crystals of perforin according to claim 1 comprising:
(i) solubilising perforin, and
(ii) adding a crystallisation solution to induce precipitation of crystals,
wherein the solution pH is between 6.4 and 8.0 and the solution temperature is 20±5° C.

3. The method for isolating crystals of perforin according to claim 1 comprising the further step of;
(iii) flash freezing the crystals, optionally in the presence of a cryoprotectant
wherein the resultant crystals are suitable for data collection at least by x-ray crystallography.

4. The method for isolating crystals of perforin according to claim 1 comprising the further step of:
(iv) subjecting expressed perforin to an initial purification step.

5. The method for isolating crystals of perforin according to claim 1 comprising the further step of:
(v) back-soaking the perforin crystallised from solution at pH 6.4 to 8.0 and 20±5° C., in a heavy metal protectant solution
wherein the back-soaked crystals are suitable for data collection at least by x-ray crystallography.

6. The method according to claim 1 wherein the perforin is chosen from the group comprising, native perforin, non-oligomerising perforin mutant, or other mutants designed with reference to the coordinates of Table 1 herein.

7. Perforin crystals prepared according to claim 1.

8. Perforin derivatives prepared using the perforin crystals of claim 7.

9. (canceled)

10. A method for screening molecules or molecular complexes for anti-perforin activity comprising the steps of:
(a) purifying perforin according to the method of the present invention,
(b) characterising an active site cavity;
(c) identifying candidate molecules or molecular complexes that interact with at least part of the active site cavity; and
(d) obtaining or synthesising said candidate molecule or molecular complex.

11. A method for screening molecules or molecular complexes for anti-perforin activity comprising the steps of:
(a) purifying perforin according to the method of the present invention;
(b) characterising the pore of the perforin;
(c) identifying candidate molecules or molecular complexes that interact with at least part of the pore; and
(d) obtaining or synthesising said candidate molecule or molecular complex.

12. The method for isolating crystals of perforin according to claim 2 comprising the further step of;
(iii) flash freezing the crystals, optionally in the presence of a cryoprotectant
wherein the resultant crystals are suitable for data collection at least by x-ray crystallography.

13. The method for isolating crystals of perforin according to claim 12 comprising the further step of:
(iv) subjecting expressed perforin to an initial purification step.

14. The method for isolating crystals of perforin according to claim 13 comprising the further step of:
(v) back-soaking the perforin crystallised from solution at pH 6.4 to 8.0 and 20±5° C., in a heavy metal protectant solution
wherein the back-soaked crystals are suitable for data collection at least by x-ray crystallography.

15. The method according to claim 14 wherein the perforin is chosen from the group comprising, native perforin, non-oligomerising perforin mutant, or other mutants designed with reference to the coordinates of Table 1 herein.
